(19) United States
(12) Patent Application Publication
(10) Pub. No.: US 2016/0131724 A1
(43) Pub. Date: May 12, 2016

(54) MRI SCANNER BORE COVERINGS

(71) Applicant: OFFICE OF TECHNOLOGY TRANSFER, NATIONAL INSTITUTES OF HEALTH, Rockville, MD (US)

(72) Inventors: Robert S. Balaban, Rockville, MD (US); Robert J. Lederman, Rockville, MD (US); Michael Hansen, Rockville, MD (US); Anthony Faranesh, Rockville, MD (US); Kanishka Ratnayaka, Rockville, MD (US)

(21) Appl. No.: 14/899,480

(22) PCT Filed: Jun. 19, 2014
(86) PCT No.: PCT/US2014/043268

§ 371 Date: Dec. 17, 2015

Related U.S. Application Data
(60) Provisional application No. 61/836,817, filed on Jun. 19, 2013.

(51) Int. Cl. G01R 33/31; A61B 5/055

(52) U.S. Cl. CPC G01R 33/31; A61B 5/055; A61B 2503/04

(57) ABSTRACT

A bore covering for sealing an MRI scanner bore to create a controlled environment within the MRI scanner bore is disclosed. The bore coverings used with climate control devices may create an incubator-like environment to allow for improved preparation, control, and imaging of infants. A system and kit including one or more bore coverings, and a method of using the bore coverings to create a controlled environment within the MRI scanner bore are also described.

### CROSS-REFERENCE TO RELATED APPLICATIONS

The present application is a PCT application of U.S. Provisional Patent Application No. 61/836,817, entitled “MRI SCANNER BORE COVERINGS” filed Jun. 19, 2013, the contents of which are incorporated by reference in its entirety herein.

### FIELD OF THE INVENTION

Aspects of the present invention relate to devices and methods related to sealable coverings for an MRI scanner bore. In particular, aspects of the invention relate to devices and methods for creating a controlled environment within an MRI scanner bore to facilitate the imaging of infants.

### BACKGROUND OF THE INVENTION

Magnetic Resonance Imaging (MRI) is an imaging technique used to image the internal structures of the body. An MRI scanner consists of a large magnet and coils to create a magnetic field and produce the image. The magnet and coils surround the MRI scanner bore of the MRI scanner, within which the patient is situated during an MRI scanning procedure. MRI scanner bores may have varying shapes and sizes, and varying bore opening shapes and sizes, depending on the design of the MRI scanner. Smaller bore openings may be associated with a traditional “closed” bore scanner; wide bore and open bore scanners may include wider or longer bore openings. In some applications, radio frequency (RF) receive coils/MRI coils may be placed on or near the patient to enhance the MRI signal in a particular area of interest. The closer the coils are situated to the patient or area of interest, the more enhancement of the image quality. RF receiver coils are typically available in different shapes and sizes to conform to different body areas of interest and different-sized patients.

MRI scanning of small infants may be particularly challenging for a variety of reasons. The MRI scanning procedure may be time consuming and the infants may be anesthetized. During the MRI scanning procedure, the atmosphere around the infant may be temperature-controlled to prevent hypothermia in the infant. Further, a number of RF receiver coils (RF/MRI coils) may be situated on the infant to enhance image quality.

A number of MRI-compatible incubators integrate incubator functionality and MRI signal receiver functionality. These MRI-compatible incubators typically include rigid boxes within which the infant is placed during the MRI scanning procedure; MRI coils are typically incorporated as part of the rigid box structure to enhance image quality. Although these MRI-compatible incubators may keep the infant warm during the MRI scanning procedure to some degree, it is relatively time consuming to place the infant in an MRI-compatible incubator. In additional, the MRI coils are rarely situated optimally to enhance image quality. In particular, the use of existing MRI-compatible incubators typically results in relatively poor body and heart imaging in infants. The cost of existing MRI-compatible incubators may be relatively high (−$50k-$100k or more) despite their limited effectiveness.

Therefore, there is a need for a device and method to create an incubator-like environment within an MRI scanner bore without the use of dedicated MRI-compatible incubators, and to provide the capability to enhance the image quality resulting from MRI scans of infants.

### BRIEF SUMMARY OF THE INVENTION

In one aspect, a bore covering for creating a controlled environment within an MRI scanner bore is provided. The bore covering includes a covering sheet with opposed outer and inner surfaces and a fastening means attached to the inner surface. The inner surface is positioned over a bore opening of the MRI scanner bore and the fastening means is reversibly attached to an exposed edge of the bore opening to form a seal over the bore opening. The attachment means may be situated near an outer edge of the bore covering and may extend completely around the outer edge, forming a continuous closed shape. The attachment means may also include one or more gaps around the outer edge, and the one or more gaps may be distributed around the outer edge. The attachment means may include one gap for accessing the MRI scanner bore from outside the bore covering. The bore covering may also include at least one opening formed within the covering sheet and extending from the outer surface to the inner surface. The at least one opening may include a first opening operatively connected to a climate control device and/or a second opening forming a vent to exhaust air out of the MRI scanner bore. The vent may include a valve to exhaust an amount of air out of the MRI scanner bore when a bore air pressure exceeds a predetermined level. At least a portion of the covering sheet is transparent and may include a plastic material. The fastening means may include a pressure-sensitive adhesive, which may be chosen from a weak-tack adhesive and a low-tack adhesive.

In another aspect, a system for creating a controlled environment within an MRI scanner bore is provided that includes at least one bore covering. Each bore covering may include a covering sheet with opposed outer and inner surfaces, a fastening means attached to the inner surface and at least one opening formed within the covering sheet and extending between the outer and inner surfaces. The system may also include at least one climate control device for regulating at least one environmental parameter within the MRI scanner bore. The at least one climate control device may be operatively connected to the at least one opening of the at least one bore covering. Each of the at least one bore coverings may be sealed over a bore opening to seal the MRI scanner bore from the outside environment. The system may further include a controller operatively connected to the at least one climate control device to control the operation of the at least one climate control device. In addition, the system may include at least one environmental sensor positioned within the MRI scanner bore and operatively connected to the controller. The controller may process each environmental parameter measured by the at least one environmental sensor and may modulate the operation of the at least one climate control device to maintain each environmental parameter to within a predetermined range.

Other aspects of the invention are described in detail below.

### BRIEF DESCRIPTION OF THE DRAWINGS

The following figures illustrate various aspects of the invention.

FIG. 1A is a top view showing the outer surface of a bore covering.

FIG. 1B is a bottom view showing the inner surface of a bore covering.

FIG. 2 is an isometric side view illustrating the bore coverings installed over the openings of an MRI scanner bore.

FIG. 3 is a bottom view showing the inner surface of a bore covering.

FIG. 4 is a bottom view showing the inner surface of a bore covering.

FIG. 5 is a bottom view showing a bore covering with a circumferential perforation.

FIG. 6 is a bottom view showing a bore covering with a perforation along a chord of the bore covering.

FIG. 7 is a schematic diagram illustrating a system for providing a climate-controlled MRI scanner bore.

Corresponding reference characters and labels indicate corresponding elements among the views of the drawings. The headings used in the figures should not be interpreted to limit the scope of the claims.

### DETAILED DESCRIPTION

Provided herein are devices, systems, and methods for sealing an MRI scanner bore and providing a controlled environment within the MRI scanner bore. The devices, systems, and methods in various aspects may include one or more bore coverings that may be sealed over one or more bore openings of the MRI scanner to provide an incubator-like controlled environment within the MRI scanner bore and eliminate the need for the use of a dedicated MRI-compatible incubator to accomplish MRI scanning of infants. In addition, the devices, systems, and methods in various aspects may also facilitate the placement of one or more RF receiver coils in close proximity to an infant during an MRI scanning procedure to enhance image quality.

The use of one or more bore coverings to seal the MRI scanner bore may transform the MRI scanner bore itself into a climate-controlled incubator for infant patients. The one or more bore coverings situated over the bore openings may essentially seal off the bore volume of the MRI scanner from the surrounding environment. Through a series of specialized openings or ports formed within the one or more bore coverings, climate-controlled air may be introduced into the MRI scanner bore resulting in a balanced equilibrium between air exchange and maintenance of internal pressure within the sealed bore volume. The air exchange may provide the capability to maintain the desired characteristics of the controlled environment within the bore volume including, but not limited to: temperature, humidity, oxygen content, and any other known characteristic of the controlled environment.

The one or more bore coverings may be easily removed and resealed over the bore opening to provide the capability to quickly and easily access the MRI scanner bore to make minor adjustments within the MRI scanner bore including, but not limited to: repositioning the patient and/or the placement of RF receiver coils. The one or more bore coverings may further include one or more viewing ports to provide for telemetry and/or visual monitoring of the patient in the sealed bore volume of the MRI scanner.

In another aspect, a system for providing a controlled environment within an MRI scanner bore may be provided that may include the one or more bore coverings operatively coupled to one or more climate control devices. In an additional aspect, the system may further include one or more sensors to monitor the environmental conditions within the sealed MRI scanner bore and/or to monitor one or more physiological states of the patient including, but not limited to core temperature, blood oxygen concentration, or respiration rate. In another additional aspect, the system may further include a feedback controller to modulate the operation of the one or more climate control devices; to maintain one or more of the sensor-measured climate conditions and/or to maintain one or more physiological states within a desired range or ranges.

Detailed descriptions of various aspects of the one or more bore coverings, systems including the one or more bore coverings, kits including the one or more bore coverings, and methods of using the one or more bore coverings to provide a controlled environment within an MRI scanner bore are provided herein below.

### I. Bore Coverings

In various aspects, one or more bore coverings may be sealed over the bore openings to provide a controlled environment within the sealed volume of the MRI scanner bore during an MRI scanning procedure. In an aspect, the bore coverings may be provided in the form of sterile single-use sheets that include adhesive on one side for attachment over the bore openings. After an MRI scanning procedure, the bore coverings may be detached and discarded, quickly rendering the MRI scanner ready for the next patient. Because the bore covering is situated in close proximity to the MRI scanner bore during use, the bore covering may be constructed of MRI-compatible materials to reduce the interference of the bore coverings with the operation of the MRI scanner.

Existing procedures to prepare an infant for MRI scanning typically involve situating the infant within a dedicated insertable MRI compatible incubator with integrated RF coils and then situating the incubator within the MRI scanner bore. The use of the bore coverings in various aspects may provide for improved preparation procedures associated with performing an MRI scanning procedure on an infant patient. Because the use of the bore coverings obviates the need for an insertable incubator, the preparation procedures are greatly simplified. The use of bore coverings may further allow enhanced MRI signal reception because RF coils may be situated in closer proximity to the patient. In addition, the bore coverings in various aspects may also permit quick access to the patient within the MRI scanner bore for a variety of actions including, but not limited to: adjusting the patient's position, adjusting the placement of RF coils on the patient, responding in the case of an emergency or to enable interventional procedures on an infant patient.

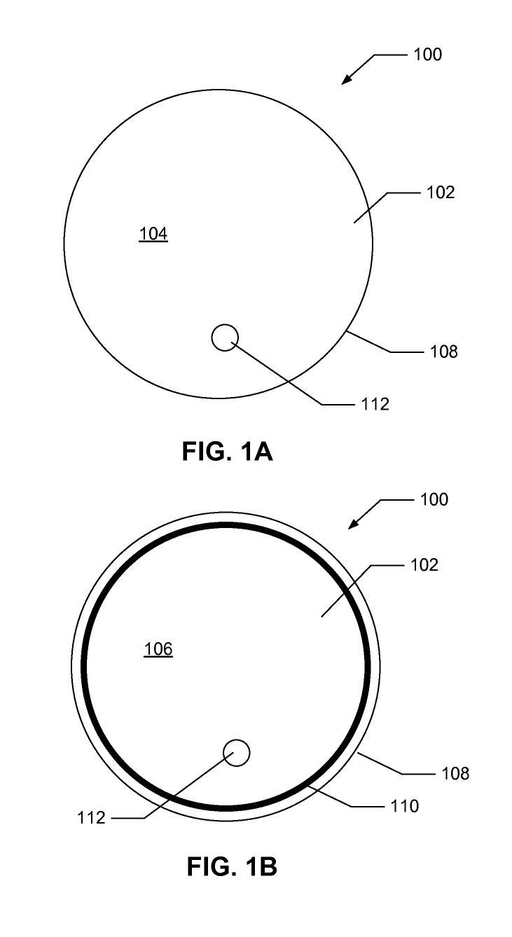

A top (outside) view and bottom (inside) view of a bore covering 100 in one aspect is shown in FIGS. 1A and 1B, respectively. In this aspect, the bore covering 100 may include a covering sheet 102 with an outer surface 104, an opposed inner surface 106 and an outer edge 108. The outer edge 108 may be dimensioned to be slightly larger than a bore opening (not shown) over which the bore covering 100 is to be sealed.

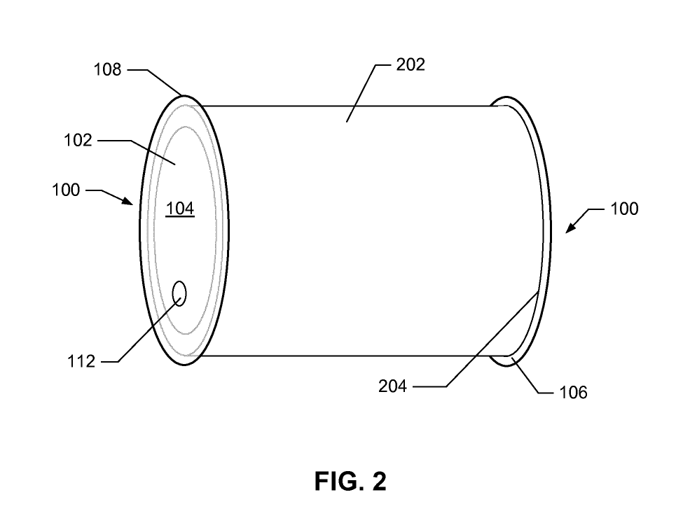

Referring to FIG. 2, when the bore covering 100 is sealed over a bore opening, the inner surface 106 is situated in contact with the exposed edge 204 of the bore opening and the outer surface 104 is situated opposite to the inner surface 106 and facing away from the bore opening. Referring back to FIG. 1B, a fastening means 110 may be attached to a region of the inner surface 106 that may contact the exposed edge of the bore opening to form a reversible sealed attachment between the bore covering 100 and the bore opening. By way of non-limiting example, the fastening means 110 may be a low tack adhesive to provide for quick removal and resealing in order to facilitate quicker access to the bore volume for adjustments or other manipulations of the infant and/or RF receiver coils within the bore volume.

The bore covering 100 may further include one or more openings 112 formed within the covering sheet 102 and extending from the outer surface 104 to the inner surface 106 to provide a conduit for the introduction of climate-controlled air into the bore volume during use. The openings 112 may further include attachment fittings (not shown) including, but not limited to air inlets to which a climate control device may attach.

a. Bore Covering Materials and Functional Properties

In various aspects, the bore coverings may be constructed using any suitable material without limitation. The bore covering may be constructed of a flexible or stiff material. In one aspect, the bore covering may be flexible to allow ease of handling during installation, to provide compact storage and transport capabilities, and to render the bore covering easily disposable. In another aspect, the bore covering may optionally be constructed of a stiffer material to provide a more robust structure and support for associated climate control devices, and/or to provide for repeated use.

The bore covering may provide an airtight seal over the bore opening to control the environment within the MRI scanner bore. To this end, the bore covering may be constructed from a relatively non-porous material and may further include an air-tight attachment means including, but not limited to an adhesive strip, described in further detail herein below, to attach the bore covering to the bore opening.

In another aspect, the bore covering material may include a polymer, without limitation. Polymers may allow the bore covering to be flexible, disposable, transparent, and/or MRI compatible. The material of the bore covering may include, but is not limited to, thermoplastics such as polyethylenes or linear low-density polyethylene (LLDPE), polypropylene, polystyrene, polyvinyl chloride (PVC), polytetrafluoroethylene (PTFE), and combinations thereof. The bore covering material may further contain additive polymers to enhance adhesiveness including, but not limited to polyisobutene (PIB), poly[ethylene-vinylacetate] (EVA) copolymer, and any other polymer that may increase the adhesiveness of the material. In another aspect the bore covering material may include thermosetting plastics including, but not limited to polyurethane.

In an aspect, the bore covering may be transparent. A transparent bore covering or portion of the covering may allow for telemetry and/or visual monitoring of the patient within the MRI scanner bore. Some visualization of the patient may be provided so that the doctor, parent, and/or other medical professional may be able to visually monitor the patient during an MRI scanning procedure. Additionally, a transparent bore covering may allow the transmission of infrared light for radiative heating of the bore volume and/or patient within the MRI scanner bore.

In one aspect, at least a portion of the covering may be transparent. The bore covering may include a transparent window for monitoring the patient and/or applying radiative heat. In another aspect, the bore covering may be adjustably transparent. In this aspect, the bore covering may change opacity in response to heat, light, or other signals. In an additional aspect, the bore covering may be opaque, and the patient may be monitored by removing at least part of the bore covering.

The bore covering may have insulative properties in various aspects. Insulation provided by the material of the bore covering may facilitate temperature control within the bore volume. In one aspect, the insulative properties of the bore covering may be imparted via the composition and/or thickness of the material used to construct the bore covering. The bore covering may include plastics having different R values, where the R value is the measure of thermal resistance known in the art. In an aspect, the bore covering may be constructed of a material having a relatively high R value to impart relatively high insulation effectiveness to the bore cover. In another aspect, the bore covering may be constructed using a relatively thick material to impart a relatively high insulation effectiveness to the bore covering.

In various aspects, the bore covering may have a uniform or non-uniform thickness. In one aspect, the bore covering may have a uniform thickness, in which the material may be relatively thin to reduce the weight, storage volume, and/or ease of use as described herein above, while performing the desired functions of the bore covering. Some of these desired functions may include, but are not limited to, easy removal from packing materials, easy installation and sealing over the MRI scanner bore, durability for repeated installation and removal, sufficient insulative properties, sufficient strength to support connections to any associated climate control equipment via the one or more openings in the bore covering.

In other aspects, the bore covering may possess a non-uniform thickness. In one aspect, the bore covering with a non-uniform thickness may include one or more locally reinforced regions. For example, the bore covering may be thinnest near the center of the covering situated near the central axis of the MRI scanner bore, and may be locally thickened near the outer perimeter of the bore covering to reinforce the region where the covering adheres to the bore opening. This reinforcement may allow for easier removal or reapplication of the bore covering. In another aspect, the bore covering may be locally reinforced near openings in the bore covering in a region where climate control equipment may attach or pass through the openings in the bore covering.

b. Attachment Means

In various aspects, the bore covering may include an attachment means to sealably attach the bore covering to the bore opening of an MRI scanner. The MRI scanner bore may be sealed at all bore openings to create a sealed, closed environment within the MRI scanner bore. Non-limiting examples of suitable attachment means include a pressure-sensitive adhesive, Velcro, ties, snaps, and any other adhesive or device that may attach the bore covering to the bore opening of the MRI scanner. In one aspect, the attachment means is a pressure-sensitive adhesive.

In one aspect, the attachment means may create a complete and air-tight seal between the bore covering and the bore opening. The attachment means may be sufficient to maintain this seal around the bore opening, even with a pressure differential between the scanner pore pressure and the pressure outside of the MRI scanner bore. In another aspect, the adhesive composition may include a weak tack adhesive or a low tack adhesive to allow repeated removal and resealing of the bore covering from/to the bore opening with relative ease. The attachment means may further permit the bore covering to be removed without leaving an adhesive residue on the MRI scanner. The attachment means may also permit quick access to the patient in an emergency situation. The attachment means may further be non-toxic and sterile, to facilitate safe use in the presence of infant patients in a medical setting.

The attachment means may include any suitable adhesive composition without limitation. Non-limiting examples of suitable adhesive compositions include: acrylic adhesives, rubber adhesives, nitrile adhesives, vinyl adhesives, hot-melt pressure sensitive adhesives, and any other adhesive capable of sealably attaching the bore covering to the MRI scanner bore. Non-limiting examples of rubber adhesives include butyl rubber, natural rubber, and silicone rubber adhesives. Non-limiting examples of hot-melt pressure sensitive adhesives include ethylene-vinyl acetate (EVA) with a high vinyl acetate content or styrene block copolymers (SBC).

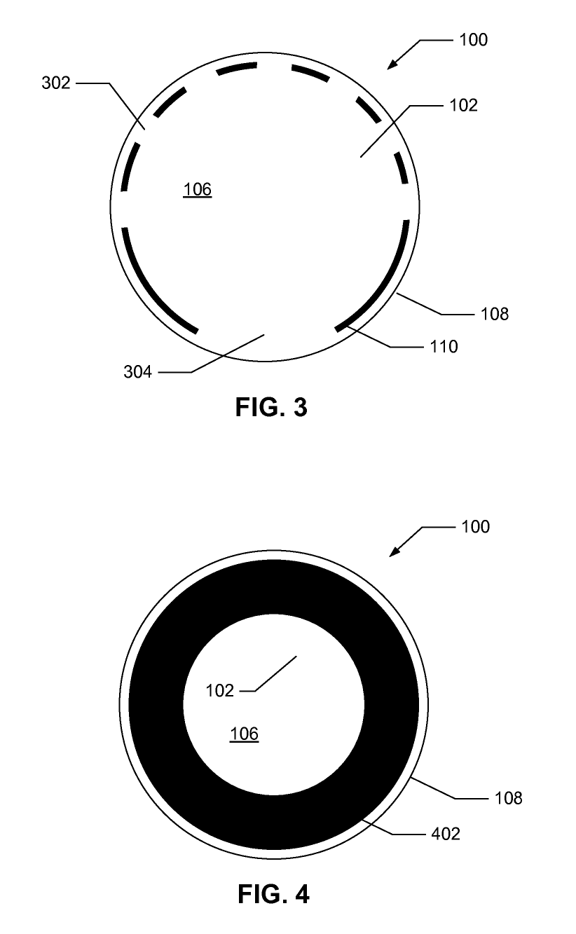

The attachment means may be attached to at least one side of the bore covering. The attachment means may be attached near the entire outer edge of the bore covering to form a continuous closed shape including, but not limited to the closed circular shape of attachment means 110 illustrated in FIG. 1B. In various aspects, the attachment means may be attached near the outermost edge of the bore covering. In an aspect, the attachment means may extend over a portion of the perimeter of the bore covering, as illustrated in FIG. 3. For example, the attachment means 110 may include one or more gaps 302 to allow for venting of the MRI scanner bore during use. In another aspect, the attachment means 110 may include a more extensive gap 304 to allow for the insertion of ducts or other conduits associated with the one or more climate control devices into the bore opening of the MRI scanner device.

The attachment means may also be situated within a region surrounding the one or more openings in the bore coverings. In one aspect, the attachment means around the one or more openings may provide an air-tight seal of the bore covering around the duct or conduit passing through each opening in the bore covering. In addition, the attachment means situated around the one or more openings of the bore covering may provide easier access and adjustability to the openings, and may further allow an unused opening to be sealed if desired.

In various other aspects, there may be a non-adhesive and removable protective layer situated over the attachment means regions of the bore covering. In an aspect, this protective layer may maintain the tack of the adhesive during storage and may further prevent self-adhesion of the bore covering during handling and installation. The protective layer may be removed when the bore covering is ready to be applied, leaving the adhesive regions exposed for attachment to the bore opening. In an aspect, the protective layer may be provided as a single piece to simplify the removal of the protective layer during the installation of the bore covering. In another aspect, the protective layer may be provided as two or more non-continuous pieces to allow the removal of a portion of the protective layer during handling or installation while leaving the remaining portion of the protective layer situated over a portion of the fastening means. For example, the upper half of a protective layer may be removed to adhere the upper portion of the bore covering to a bore opening of an MRI scanner, and the lower half of the protective layer may be removed to align and adhere the remaining half of the bore covering only after the upper portion of the bore covering has been adhered in place.

c. Size and Shape of Bore Covering

The bore covering may any size and shape to accommodate a variety of different sizes and shapes of bore openings of MRI scanners without limitation. In an aspect, the shape of the bore covering may include, but is not limited to, circular, rounded, square, rectangular, or any other shape capable of covering a bore opening. In another aspect, a bore covering size and/or shape may be specific to a particular model, brand, or type of MRI scanner.

In an aspect, the bore covering may be an essentially circular shape to cover the bore opening as illustrated in FIG. 1. In one aspect, the patient table may be removed during the installation of the bore covering. In various aspects, the diameter of a circular bore covering may range from about 40 cm to about 80 cm. In various other aspects, the diameter of a circular bore covering may range from 40 cm to 50 cm, from 45 cm to 55 cm, from 50 cm to 60 cm, from 55 cm to 65 cm, from 60 cm to 70 cm, from 65 cm to 75 cm, and from 70 cm to 80 cm. In another aspect, the bore covering may be essentially circular with a segment removed to accommodate an attached patient table.

In another aspect, the bore covering may be essentially rectangular to accommodate a variety of different sizes and shapes of MRI scanner bores. In this aspect, the length or width of the rectangular bore covering may range from about 40 cm to about 240 cm. In various aspects, the rectangular bore covering length or width may range from 40 cm to 60 cm, from 50 cm to 70 cm, from 60 cm to 80 cm, from 70 cm to 90 cm, from 80 cm to 120 cm, from 100 cm to 140 cm, from 120 cm to 160 cm, from 140 cm to 180 cm, from 160 cm to 200 cm, from 180 cm to 220 cm, and from 200 cm to 240 cm. In another aspect, the bore covering may be essentially rectangular with a segment removed to accommodate an attached patient table. A rectangular bore covering may be used to seal the bore opening of an open MRI scanner in one aspect.

The size and/or shape of the bore covering may be adjustable to accommodate a variety of different sizes or shapes of MRI scanner bores in various aspects. In one aspect, a band 402 of the attachment means may extend inward from the perimeter of the bore covering to facilitate adhesion to a variety of bore diameters or open MRI sizes, as illustrated in FIG. 4.

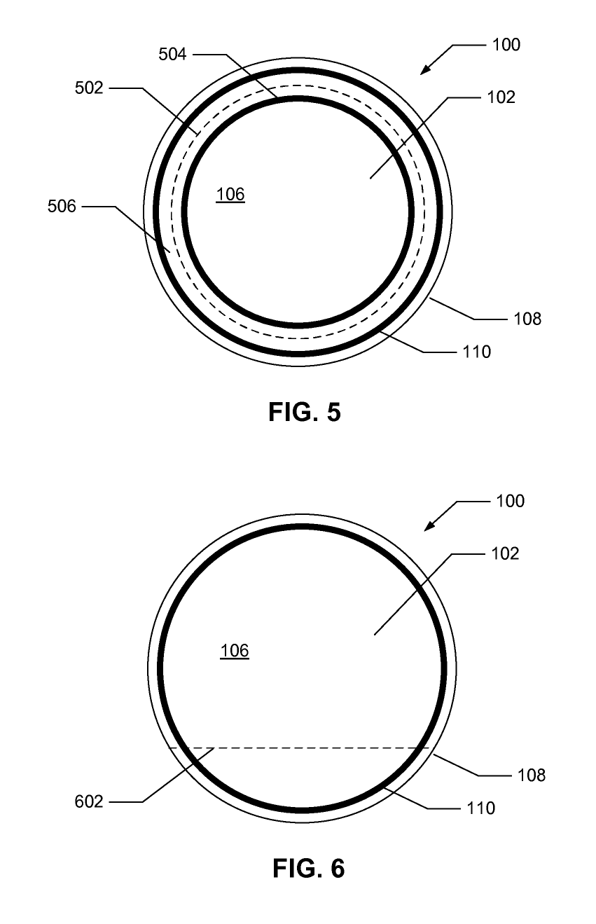

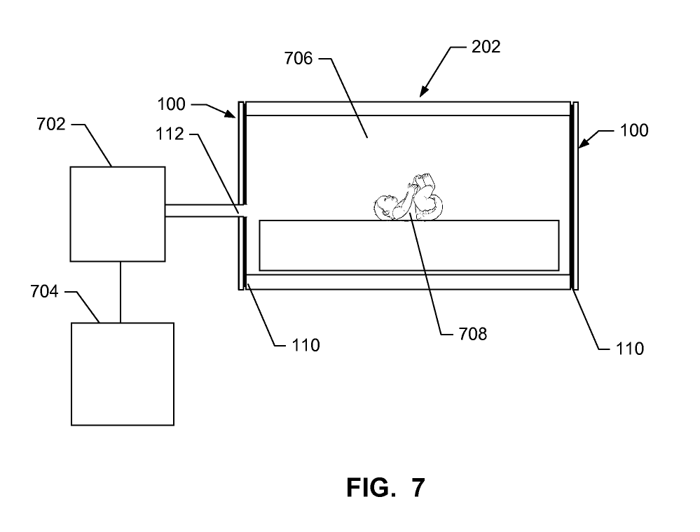

The bore covering may further include one or more perforations to facilitate the adjustment in the size and/or shape of the bore covering to accommodate different bore openings associated with different MRI scanners in various embodiments. As illustrated in FIG. 5, the bore covering 100 may include perforations 502 in the form of concentric circles within material of the covering sheet 102 to allow for removal of excess covering material between the outer edge of the bore covering and the region of the bore covering to be adhered to the bore opening. In this aspect, the bore covering 100 may further include an inner attachment means 504 to provide adhesion to the smaller bore opening once the outer portion 506 and outer attachment means 110 of the bore covering 100 are removed by separating along the perforation 502.

In another aspect, the bore covering 100 may include chord-wise perforations 602 along at least one chord of a circular bore covering 100 to allow removal of a segment to accommodate an attached patient table, as illustrated in FIG. 6. In yet another aspect, a rectangular bore covering may be similarly perforated across the width or height of the bore covering to accommodate different bore opening sizes and shapes. The rectangular bore covering may include multiple perforations across the full width at various heights, for example, in a manner similar to the perforations provided within adjustable-sized paper towels.

The bore covering may include foldable outer portions to reduce the size of the covering to fit various sized bore openings in various aspects. In one aspect, the bore covering may include an adhesive region that may be exposed to hold a folded region in place. For example, a circular bore covering may be folded along a radius to reduce the outer perimeter of the bore covering. In another aspect, a rectangular bore covering may be folded along the length and/or width of the bore covering to reduce the height and/or length of the bore covering.

d. Openings in Bore Covering

In various aspects, the bore covering may include one or more openings to provide access to the MRI scanner bore when the bore covering is sealed in place. For example, a duct or conduit of a climate control device may connect to an opening within the bore covering. In another example, an opening may provide quick and easy access to the patient within the scanner bore without need to remove the bore covering.

In one aspect, the one or more openings may be provided in the form of a hole or slit within the bore covering, and may optionally include a flap or duct integrated into the bore covering. The bore covering may be locally reinforced around an opening to provide support for the connection to a climate control device to the bore covering. The openings may allow heated or humidified air, or any other desired substance to be added or removed from the sealed MRI scanner bore.

To control the environment within a sealed MRI scanner bore, the MRI scanner bore may be connected to at least one climate control device configured to control at least one environmental parameter within the MRI scanner bore. Environmental parameters that the climate control devices may control may include, but are not limited to, temperature, pressure, humidity, oxygen content, or any other parameter that may influence the environment within the bore volume. Non-limiting examples of climate control devices that may be connected to an opening include a hot air blower or other heating element, a humidifier or dehumidifier, an oxygen tank or oxygen generator, and any other known climate control device.

In one aspect, the opening may be a hole formed within the bore covering. In this aspect, the hole may be sized to be smaller than the duct or conduit of a climate control device so that the opening may stretch to accommodate and seal to the duct or conduit. The opening in this aspect may further include an adhesive situated around the perimeter of the hole to enhance the tightness of the seal. The opening may be locally thickened or reinforced as described herein above to ensure adequate sealing and support of the duct or conduit of the climate control device.

In various other aspects, the opening may be adjustable in size. The opening may be provided in the form of a slit, a hole with a flap, or any other opening with an adjustable size. The adjustable-sized opening may allow for varying sized ducts or climate control devices to be attached to the bore covering. The opening or duct may include an adhesive to enhance the tightness of the seal or be locally thickened or reinforced as described herein above. In an aspect, adhesive tape may be wrapped around the perimeter of the opening to seal the bore covering against the duct of the climate control device.

An inlet duct may be integrated into the opening of the bore covering in another aspect. In this aspect, the inlet duct may attach to a fitting associated with the output of a climate control device. In an aspect, an integrated inlet duct may be adjustable to be compatible with a variety of climate control devices. Non-limiting examples of adjustable features of an integrated inlet duct include one or more attachment fitting adaptors to accommodate one or more attachment fittings associated with different climate control devices, and regions of the duct with different sizes or shapes that may be trimmed or cut to expose a region compatible with a particular climate control device's attachment fitting. Non-limiting examples of other adjustable integrated duct features include a radiator clamp and a drill chuck-like fitting that may dilate or contract to a variety of diameters.

In an aspect, the opening of the bore covering may be adapted to attach to one particular type of climate control device. In an aspect, the bore covering may include different types of openings for different types of climate control devices. For example, one opening may be used to attach a particular climate control device, and any remaining openings may be sealed during use.

The bore covering may optionally include an opening dedicated to venting the MRI scanner bore. This opening for venting may include a valve. The valve may be non-adjustable or adjustable in various aspects. In one aspect, the opening may include a non-adjustable valve that opens when the pressure within the bore volume exceeds the external pressure by a predetermined amount, in a manner similar to a pressure relief valve. In another aspect, the opening may include an adjustable valve. Non-limiting examples of adjustable valves include valves that may be adjusted manually, valves that open and close at predetermined time intervals, valves that open in response to measured environmental parameters, and valves that may operate in any way which allows the air from inside the MRI scanner bore to be vented to the outside. The adjustable valve may be connected to a low pressure or vacuum source in one aspect; the low pressure source may pull the air from the inside of the MRI scanner bore to the outside. In another aspect, the air may flow through the valve due to the pressure differential between the MRI scanner bore and the outside.

The bore covering may also include additional openings to provide access to the MRI scanner bore before or during MRI scanning or treatment. This access opening may allow an operator to reach into the MRI scanner bore to adjust the infant, RF coils, and/or any other aspect of the system. In an aspect, the access opening may be one or more slits or flaps formed within the bore covering that may be reversibly opened, closed, and/or resealed. Adhesive or other fastening means may be situated around the perimeter of the access opening to permit resealing of the access opening. In another aspect, the access opening may be defined by perforations which may retain the bore covering in an intact condition unless the opening is formed by separating the material of the bore covering along the perforations. In this aspect, the perforated region may be separated to open an access opening initially. Adhesive or other attachment means may also be situated in the region of the access opening to seal the access opening once opened. The access openings may be locally reinforced to provide more structural support and to maintain the integrity of the bore covering during use.

In various aspects, the bore covering may include any number of openings without limitation including, but not limited to, zero, one, two, three, four, five, six, or any number of openings necessary to control the environment and/or provide access within the MRI scanner bore. In one aspect, the bore covering may lack any openings. In another aspect, the bore covering may include two, four, six, or any even number of openings to allow input and output to and from the MRI scanner bore and the climate control device used to regulate the environment.

d. Additional Features of Bore Covering

The bore covering may further include additional features that may enhance the function of the bore coverings in use. In one aspect, the bore covering may include one or more integrated sensors to monitor one or more environmental parameters within the bore volume. The integrated sensors may include, but are not limited to, sensors for temperature, humidity, or other environmental parameters. The integrated sensor may be situated on the bore covering such that an operator may monitor the environment within the MRI scanner bore once the bore covering is sealed over the bore opening. In an aspect, the integrated sensor may be a temperature strip with an LCD display for monitoring of the temperature within the MRI scanner bore.

In various other aspects, the bore coverings may be disposable to avoid cross contamination issues inherent in existing devices such as insertable MRI-compatible incubators. In addition to preventing contamination between patients, the disposable bore coverings may eliminate any time spent cleaning and sterilizing the bore coverings, and may further reduce the cost of the bore coverings. In these other aspects, the disposable coverings may be discarded after use.

### II. System for Climate-controlling an MRI Scanner Bore

In various aspects, the bore coverings may be included in a system for sealing an MRI scanner bore to create a climate-controlled environment within the MRI scanner bore. The system may convert the MRI scanner bore itself into an incubator to facilitate the performance of MRI scanning procedures on infant patients. This system for climate-controlling the MRI scanner bore may include, but is not limited to, the one or more bore coverings as described above, one or more climate control devices operatively connected to the one or more bore coverings to deliver and/or remove climate-controlled air to the MRI scanner bore, and a controller operatively connected to the one or more climate control devices to control the operation of the one or more climate control devices in order to maintain the climate within the MRI scanner bore at desired conditions.

The bore coverings may be attached along the exposed edges of the bore openings of an MRI scanner bore to completely cover the bore opening and seal the MRI scanner bore from the outside environment as described herein previously. The system may facilitate the use of RF receiver coils situated near the patient in the MRI scanner bore to improve the image quality, may provide easier access to the patient at any time in the MRI scanner bore, and may allow quicker preparation time. In one aspect, the bore coverings may be affixed over the bore openings of the MRI scanner bore once the patient is situated within the MRI scanner bore.

FIG. 6 is a schematic illustration of a system for climate-controlling an MRI scanner bore in an aspect. The bore coverings 100 are attached over the bore openings of the MRI scanner 202 via the attachment means 110. One of the bore coverings 100 may contain at least one opening 112 for connection to at least one climate control device 702. The climate control device 702 may be operatively connected to a controller 704. The controller 704 may include one or more feedback sensors (not shown) within the MRI scanner bore 706 for informing adjustments to the climate control device 702. An infant patient 708 may be placed inside the MRI scanner bore 706 prior to sealing the bore coverings 100 in place.

a. Bore Coverings

The system may include at least one bore covering similar to the bore coverings described herein previously. Each bore covering may be sealably attached over each bore opening of the MRI scanner bore. In an aspect, one bore covering may seal the MRI scanner bore from the outside environment, depending on the design of the MRI scanner. In another aspect, two bore coverings may seal the MRI scanner bore from the outside environment; one bore covering may be sealed over a bore opening at each end of a cylindrical MRI scanner bore. The system may further include an optional additional covering for inside the MRI scanner bore to provide a sterile surface for the infant patient during the MRI scanning procedure.

In various aspects, the number of bore coverings corresponds to the number of bore openings in order to provide a sealed volume within the MRI scanner. For example, an MRI scanner with a closed cylindrical bore may be sealed using a total of two circular bore coverings, one for each open end. For an open MRI scanner, the entire perimeter of the MRI scanner bore may use two or more bore coverings to sufficiently seal the MRI scanner bore.

The bore coverings may be attached to the MRI scanner using the attachment means situated on one side of the bore covering as described herein previously. In an aspect, the attachment means may be an adhesive. The attachment means may allow quick removal of the bore covering, but still provide a seal around the opening of the MRI scanner bore to isolate the environment inside the MRI scanner bore from the environment outside the MRI scanner bore. After an MRI scanning procedure, the bore coverings may be discarded or autoclaved and the MRI scanner may be immediately ready for the next patient.

b. Climate Control Devices

The system may include one or more climate control devices operatively connected to one or more bore coverings to deliver and/or remove climate-controlled air to/from the MRI scanner bore. In various aspects, the bore covering may be connected to at least one climate control device through at least one opening on a bore covering to adjust the environment within the MRI scanner bore as described herein previously. To control the environment within a sealed MRI scanner bore, the at least one climate control device may control at least one environmental parameter within the MRI scanner bore. Non-limiting examples of environmental parameters that the climate control device may control include temperature, pressure, oxygen, humidity, or any other parameter that may affect the environment within the MRI scanner bore.

The climate control device may be an MRI compatible device to ensure reliable operation in close vicinity to the MRI scanner. In an aspect, the climate control device may not be MRI compatible if it is situated well away from the MRI scanner. In this aspect, the climate control device may be connected to the bore covering using MRI compatible ducts or tubes. Non-limiting examples of suitable climate control devices that may be connected to an opening in the bore covering include a hot air blower, an infrared lamp or other heating device; a humidifier or dehumidifier; an oxygen tank or oxygen generator; and any other suitable climate control device.

The one or more climate control devices may produce an incubator-like environment within the MRI scanner bore to minimize patient distress during an MRI scanning procedure. In an aspect, the climate control device may provide heating and/or temperature control for the atmosphere in the MRI scanner bore. In an aspect, temperature and humidity controlled air from a climate control device may be introduced into the MRI scanner bore, generating a balance between air exchange and internal pressure. The climate control device may use convective heating or radiative heating to heat and/or control the temperature within the MRI scanner bore. The temperature of the hot air may be manually controlled by a doctor, nurse, anesthesiologist, or any other medical professional based on the patient core temperature.

A climate control device may produce or regulate humidity within the MRI scanner bore. In an aspect, water vapor may be added to the heated air in a convective heating climate control device. In another aspect, water vapor may be introduced independently of the heating or temperature climate control device. Non-limiting examples of climate control devices for controlling humidity may include an evaporator or an ultrasonic vaporizer.

A climate control device may deliver oxygen to the MRI scanner bore. In an aspect, the climate control device may add oxygen to the heated air in a convective heating climate control device. In another aspect, oxygen may be introduced independently of the heating climate control device. Non-limiting examples of climate control devices for introducing or controlling oxygen may include an oxygen tank or an oxygen generator.

A climate control device may by used to introduce other substances into the MRI scanner bore and/or into the patient. The climate control device may be used to introduce compounds including, but not limited to medicines, anesthesia, or MRI contrast agents, into the sealed MRI scanner bore.

c. Controller

The system may include a controller to control the operation of one or more of the climate control devices in various aspects. In one aspect, the controller may be used to maintain or adjust environmental parameters within the sealed MRI scanner bore by controlling the operation of the one or more climate control devices. One or more environmental sensors may be operatively connected to the controller; the environmental sensors may be situated within the MRI scanner bore. The one or more environmental sensors may permit constant appropriate adjustment of the environment by monitoring the environmental conditions within the MRI scanner bore for the controller, which then may adjust the climate control devices appropriately.

In another aspect, one or more physiological sensors may be operatively attached to the controller and situated on or near the patient within the MRI scanner bore. In this other aspect, the one or more physiological sensors may monitor one or more physiological conditions of the patient, including, but not limited to, a respiration rate, a heart rate, a blood oxygen level, a core or other body temperature, a patient movement, or any other relevant physiological condition. In this other aspect, the controller may modulate the operation of the one or more climate control devices based on feedback received from the one or more physiological monitors.

The controller may include manual controls in one aspect. The operator may use the controller to manually adjust the levels of the climate control devices. In this aspect, the system may further include one or more environmental sensors and/or physiological sensors similar to those described herein above. The output of the sensors may be monitored by the operator of the system, and the manual controls of the controller may be adjusted by the operator to maintain the output of the sensors within desired ranges.

In another aspect, the controller may be an automatic controller. In one aspect the automatic controller may be an open loop controller to operate the climate control devices according to a predetermined schedule. In another aspect, the automatic controller may be a closed loop controller to operate the climate control devices based on information received from one or more environmental or physiological sensors operatively connected to the controller as described herein above.

The environmental sensors or physiological sensors may be operatively attached to a display and/or recording system to allow the operator to monitor the environmental parameters or physiological conditions of the patient being measured. This display may further inform an operator if a manual controller or manual valve is being used within the system, as described below.

d. Operation of System

The climate control devices may be operatively connected to the MRI scanner bore through openings in the bore coverings. The bore coverings may provide enough openings to allow the connection of the climate control devices, or input, and venting, or output from the MRI scanner bore.

The coverings may be adapted to receive a connection from one or more climate control devices, as described herein above. In another aspect, the openings in the bore coverings may include an integrated duct that may be connected to one or more climate control devices, as described above.

Climate-controlled air may enter and excess air may be vented from the MRI scanner bore. The resulting air movement may prevent the depletion of oxygen within the MRI scanner bore. The introduction of the air and the venting may occur through one or more openings within a single bore covering. In another aspect, the input and venting may occur via openings on different bore coverings. Without being limited to a particular theory, this aspect may create complex air flow patterns within the MRI scanner bore. For example, without being limited to a particular theory, if air is introduced at one end of an MRI scanner bore and vented at an opposite end of the MRI scanner bore via openings in two different bore coverings, a unidirectional air flow pattern may be induced that moves the air from one end of the MRI scanner bore to the other.

The venting of air from the MRI scanner bore may be passive or active in various aspects. In one aspect, passive venting may occur through small leaks anywhere within the sealed bore coverings as described herein above. In another aspect, the bore covering may include a dedicated vent opening. This vent opening may be sized to provide a predetermined back-pressure within the MRI scanner bore. In this aspect, the bore covering may optionally be provided with a passive valve that initiates venting when pressure within the MRI scanner bore exceeds a predetermined pressure. In this aspect, the passive valve may be a relief valve.

The system may include an active valve within the bore covering for venting the MRI scanner bore. The vent opening on the bore covering may include a controllable valve which may allow excess air to be pushed out by pressure within the MRI scanner bore. In an aspect, the controllable valve may be manually adjusted by the operator. In another aspect, the controllable valve may open and/or close at predetermined time intervals. In yet another aspect, the controllable valve may open in response to measurements of environmental parameters including, but not limited to pressure, temperature, or humidity. The controllable valve may be connected to the controller in an aspect.

The vent opening may be operatively connected to a low pressure or vacuum source with a controllable valve. The low pressure source would effectively suck out excess air in the MRI scanner bore. In an aspect, the controllable valve connected to the low pressure source may be manually adjusted by the operator. In another aspect, the controllable valve connected to the low pressure source may open and/or close at predetermined time intervals. In yet another aspect, the controllable valve connected to the low pressure source may open in response to measurements of environmental parameters including, but not limited to pressure, temperature, or humidity. The controllable valve may be connected to the feedback sensor and/or the controller.

### III. Bore Covering Kit

In various aspects, a kit for creating a controlled environment within an MRI scanner bore may be provided. The kit may include at least one bore covering, optional coverings for inside the MRI scanner bore, optional feedback sensors, optional physiological sensors, optional climate control devices and associated connectors, and an optional controller. The kit may be used to create an incubator-like environment within an MRI scanner bore.

The kit for creating a controlled environment may include one or more bore coverings as described above. The bore coverings may be custom-sized for individual MRI scanners or may be adjustable to fit the bore opening. The bore coverings may include various openings or adaptors for connecting different climate control devices as needed.

In an aspect, the bore coverings may be packaged in sterile packaging. The sterile packaging may be in the form of a vacuum film, a sealed bag, or any other packaging capable of keeping the bore covering sterile during shipping and storage. In another aspect, the bore coverings may be autoclavable for reuse.

The openings within the bore coverings may optionally include attachments or ducts for easier attachment of climate control devices to the bore covering. The region around the bore covering may be reinforced to support the attachment of a climate control device.

The kit may optionally include one or more MRI compatible climate control devices for adjusting the environment within the sealed MRI scanner bore as described above. The climate control device may adjust the temperature, humidity, oxygen, carbon dioxide, or other environmental parameters within the MRI scanner bore. The climate control device may attach to the bore covering through an opening provided in the bore covering.

The kit may optionally include a controller as described herein above. In an aspect, the controller may be optionally attached to climate control devices. In another aspect, the controller may be optionally attached to feedback or physiological sensors. The feedback sensors may be placed within the MRI scanner bore to send a signal about the environmental parameters within the MRI scanner bore to the controller. The physiological sensors may be placed on the infant for monitoring of the infant's vital signs during the imaging process. The sensors may be operatively attached to a display and/or recording device.

### IV. Method of Use: Infant Imaging

In various aspects, the bore coverings, as well as systems and kits including the bore coverings, may be used to provide an incubator-like environment while performing an MRI scanning procedure on an infant patient. A method of using the bore coverings in an MRI scanning procedure of an infant patient may include sealing an MRI scanner bore with one or more bore coverings, connecting at least one climate control device to the MRI scanner bore through at least one opening in the bore covering, and operating the climate control devices to maintain an controlled environment within the MRI scanner bore. The bore coverings connected to at least one climate control device may create an incubator-like environment within an MRI scanner bore. The incubator-like environment may allow the imaging of an infant without the use of an external incubator. In this method, RF coils may be placed on or near the infant within the MRI scanner bore prior to starting the MRI scanning procedure. The RF coils placed on or near the infant may enhance the image quality relative to the quality of an image obtained during an MRI scanning procedure using coils within an external incubator or without any coils.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

What is claimed is:

1. A bore covering for creating a controlled environment within an MRI scanner bore, wherein the bore covering comprises a covering sheet with opposed outer and inner surfaces and a fastening means attached to the inner surface, wherein the inner surface is positioned over a bore opening of the MRI scanner bore and the fastening means is reversibly attached to an exposed edge of the bore opening to form a seal over the bore opening.

2. The bore covering of claim 1, wherein the attachment means is situated near an outer edge of the bore covering.

3. The bore covering of claim 2, wherein the attachment means extend completely around the outer edge, forming a continuous closed shape.

4. The bore covering of claim 2, wherein the attachment means include one or more gaps around the outer edge.

5. The bore covering of claim 4, wherein the one or more gaps are distributed around the outer edge.

6. The bore covering of claim 4, wherein the attachment means include one gap for accessing the MRI scanner bore from outside the bore covering.

7. The bore covering of claim 1, wherein the bore covering further comprises at least one opening formed within the covering sheet and extending from the outer surface to the inner surface.

8. The bore covering of claim 7, wherein the at least one opening comprises a first opening operatively connected to a climate control device.

9. The bore covering of claim 8, wherein the at least one opening further comprises a second opening comprising a vent to exhaust air out of the MRI scanner bore.

10. The bore covering of claim 9, wherein the vent comprises a valve to exhaust an amount of air out of the MRI scanner bore when a bore air pressure exceeds a predetermined level.

11. The bore covering of claim 1, wherein at least a portion of the covering sheet is transparent.

12. The bore covering of claim 1, wherein the covering sheet further comprises a plastic material.

13. The bore covering of claim 1, wherein the fastening means comprises a pressure-sensitive adhesive.

14. The bore covering of claim 13, wherein the pressure-sensitive adhesive is chosen from a weak-tack adhesive and a low-tack adhesive.

15. A system for creating a controlled environment within an MRI scanner bore comprising:
at least one bore covering, each bore covering comprising a covering sheet with opposed outer and inner surfaces, a fastening means attached to the inner surface and at least one opening formed within the covering sheet and extending between the outer and inner surfaces; and
at least one climate control device for regulating at least one environmental parameter within the MRI scanner bore, wherein the at least one climate control device is operatively connected to the at least one opening of the at least one bore covering;
wherein each of the at least one bore coverings is sealed over a bore opening to seal the MRI scanner bore from the outside environment.

16. The system of claim 15, further comprising a controller operatively connected to the at least one climate control device to control the operation of the at least one climate control device.

17. The system of claim 16, further comprising at least one environmental sensor positioned within the MRI scanner bore and operatively connected to the controller, wherein the controller processes each environmental parameter measured by the at least one environmental sensor and modulates the operation of the at least one climate control device to maintain each environmental parameter to within a predetermined range.
